(19) United States
(12) Patent Application Publication
(10) Pub. No.: US 2020/0238038 A1
(43) Pub. Date: Jul. 30, 2020

(54) Anesthesia machine, oxygen battery calibration system and calibration method thereof

(71) Applicants: SHENZHEN MINDRAY BIO-MEDICAL ELECTRONICS CO., LTD., Shenzhen (CN); Shenzhen Mindray Scientific Co., Ltd., Shenzhen (CN)

(72) Inventors: Xuetao WU, Shenzhen (CN); Peitao CHEN, Shenzhen (CN)

(73) Assignees: SHENZHEN MINDRAY BIO-MEDICAL ELECTRONICS CO., LTD., Shenzhen (CN); Shenzhen Mindray Scientific Co., Ltd., Shenzhen (CN)

(21) Appl. No.: 16/846,262

(22) Filed: Apr. 10, 2020

Related U.S. Application Data
(63) Continuation of application No. PCT/CN2017/107102, filed on Oct. 20, 2017.

(51) Int. Cl. A61M 16/10; A61M 16/20; A61M 16/01

(52) U.S. Cl. CPC A61M 16/104; A61M 16/208; A61M 16/01; A61M 2205/70; A61M 2202/048; A61M 2202/0208; A61M 2205/3334; A61M 2205/8206

(57) ABSTRACT

An oxygen battery calibration system can include a breathing circuit that includes an inspiratory branch, an expiratory branch, an absorption tank branch, an inspiratory one-way valve, a connection pipeline and an expiratory one-way valve. The inspiratory and expiratory branches can communicate via the connection pipeline. The inspiratory and expiratory one-way valves can be arranged respectively in the inspiratory and expiratory branches, one end of the absorption tank branch can communicate with the inspiratory branch and can be located at a front end of the inspiratory one-way valve, and the other end of the absorption tank branch can communicate with the expiratory branch and can be located at a rear end of the expiratory one-way valve. The system can also include an oxygen battery connected to the inspiratory branch, with a joint being located at a rear end of the inspiratory one-way valve, and a calibration management controller.

### CROSS-REFERENCE TO RELATED APPLICATIONS

This application is a bypass continuation of PCT App. No. PCT/CN2017/107102 filed Oct. 20, 2017, for ANESTHESIA MACHINE, OXYGEN BATTERY CALIBRATION SYSTEM AND CALIBRATION METHOD THEREOF, which is incorporated herein by reference.

### TECHNICAL FIELD

The present disclosure relates to the technical field of anesthesia equipment, and in particular to an anesthesia machine, an oxygen battery calibration system and a calibration method thereof.

### BACKGROUND

During the ventilation of anesthesia machines, different oxygen concentrations will be given according to the individual conditions of patients. In order to ensure that the oxygen concentration inhaled by a patient is within a set range, the anesthesia machine will generally be equipped with an oxygen battery in a breathing circuit to monitor the oxygen concentration of gas in real time.

Oxygen batteries commonly used in the anesthesia machines are divided into chemical oxygen batteries and paramagnetic oxygen batteries. The former realizes the measurement of oxygen concentration by means of the mechanism that oxygen molecules react with a specific chemical substance in the oxygen battery to generate a current. Different currents will be generated when different concentrations of oxygen enter the oxygen battery. Before use, the oxygen battery needs to be calibrated using a gas with known oxygen concentrations, usually at two points (such as oxygen concentrations of 21% and 100%), so as to obtain a corresponding linear relationship between the oxygen concentration and the current. When measuring the oxygen concentration in actual work, a reverse solution is carried out according to the measured current and the corresponding function relationship between the oxygen concentration and the current to obtain a current value of the oxygen concentration. During the operation of the chemical oxygen battery, as the measurement time passes, the corresponding relationship between the oxygen concentration and the current will drift due to the change in the form of the chemical substance. In order to ensure the accuracy and reliability of measurement of the oxygen concentration, the oxygen battery needs to be calibrated regularly within the service life of the oxygen battery. The paramagnetic oxygen battery is based on the paramagnetic characteristics of oxygen. A typical measurement method consists in that when a gas to be measured enters the paramagnetic oxygen battery, the gas will be sucked into a magnetic field and impact on the internal physical structure thereof, which causes the internal physical structure to generate a deflection moment to obtain a linear relationship between the oxygen concentration and the moment (current). During measuring the oxygen concentration in actual work, the reverse solution is carried out according to the actually measured magnitude of the current and the corresponding function relationship between the oxygen concentration and the current to obtain the current value of the oxygen concentration. The paramagnetic oxygen battery carries out the measurement by means of purely physical principles. Theoretically, there is no limit on the service life, however, usually due to factors such as movement and impact, the internal structure may be changed, causing measurement deviations. Therefore, the paramagnetic oxygen battery also needs to be calibrated usually at one point (e.g. oxygen concentration of 100%) according to actual requirements.

At present, most of the anesthesia machines on the market use a chemical oxygen battery. When the calibration is performed at the oxygen concentration points of 21% and 100%, it is usually necessary to remove the oxygen battery and place it in the air for a period of time, such as 1 to 3 minutes. Then, calibration operations are performed in a calibration procedure to acquire the current value of the current output by the oxygen battery, and the oxygen battery is then mounted back to the anesthesia machine. A fresh gas (pure oxygen) is adjusted to flush the circuit for a period of time, usually 1 to 3 minutes, and the calibration operations are then performed in the calibration procedure to acquire the current value of the current output by the oxygen battery. The earlier and later current values respectively correspond to the points where the oxygen concentrations are 21% and 100%, so as to obtain the linear relationship between the oxygen concentration and the current. The current calibration operations require manual intervention, and the operation procedure is complicated and time-consuming.

### SUMMARY

In view of this, it is necessary to provide an oxygen battery calibration system in response to the problem of the current anesthesia machine requiring manual intervention during automatic calibration of the oxygen battery, an anesthesia machine containing the above-mentioned oxygen battery calibration system is further provided, and an calibration method applied to the above-mentioned oxygen battery calibration system is provided.

The foregoing objectives are achieved by the following technical solutions. An oxygen battery calibration system may include a breathing circuit that includes an inspiratory branch, an expiratory branch, an absorption tank branch, an inspiratory one-way valve, a connection pipeline and an expiratory one-way valve, wherein the inspiratory branch and the expiratory branch communicate via the connection pipeline, the inspiratory one-way valve is arranged in the inspiratory branch, the expiratory one-way valve is arranged in the expiratory branch, one end of the absorption tank branch communicates with the inspiratory branch and is located at a front end of the inspiratory one-way valve, and the other end of the absorption tank branch communicates with the expiratory branch and is located at a rear end of the expiratory one-way valve. The oxygen battery calibration system may further include an oxygen battery connected to the inspiratory branch, with a joint being located at a rear end of the inspiratory one-way valve. The oxygen battery calibration system may additionally include a calibration management controller, the calibration management controller controlling a calibration gas to enter the inspiratory branch and flow out through the oxygen battery, the connection pipeline and the expiratory branch, and the calibration management controller performing an oxygen concentration calibration according to the calibration gas flowing through the oxygen battery.

In one embodiment, the oxygen battery calibration system further includes a bypass branch, wherein the bypass branch and the inspiratory branch are connected between the inspiratory one-way valve and the oxygen battery. The calibration management controller may control the calibration gas to enter the inspiratory branch through the bypass branch during the oxygen concentration calibration.

In one embodiment, the oxygen battery calibration system further includes a switch component, wherein the switch component is arranged in the absorption tank branch for controlling the opening and closing of the absorption tank branch. When the oxygen concentration calibration is performed, the switch component closes the absorption tank branch and the calibration gas is capable of entering the inspiratory branch.

In one embodiment, the switch component is a switch valve or an air-resistor.

In one embodiment, one end of the bypass branch is connected to a common gas outlet or a fresh gas outlet of an anesthesia machine, and the other end thereof is connected to the rear end of the inspiratory one-way valve in the inspiratory branch.

In one embodiment, an input end of the inspiratory branch communicates with a gas source module of an anesthesia machine, or a common gas outlet, or a fresh gas outlet, and the calibration management controller controls the calibration gas to enter the inspiratory branch during the oxygen concentration calibration.

In one embodiment, the calibration management controller calibrates the oxygen battery with the calibration gas having at least two different oxygen concentrations.

In one embodiment, the oxygen battery is a chemical oxygen battery.

In one embodiment, the oxygen battery is a paramagnetic oxygen battery, and the calibration management controller calibrates the oxygen battery with the calibration gas having at least one oxygen concentration.

In an example embodiment, an anesthesia machine may include an anesthetic supply device, an exhaust gas discharge device, and an oxygen battery calibration system according to any of the above technical features. One end of the inspiratory branch of the breathing circuit of the oxygen battery calibration system may communicate with the anesthetic supply device, and one end of the expiratory branch of the breathing circuit may communicate with the exhaust gas discharge device. During use of the anesthesia machine, the anesthetic supply device supplies an inspiratory gas containing an anesthetic to the breathing circuit, the inspiratory gas enters the inspiratory branch and is then supplied to a patient through the connection pipeline, and at the same time, an exhaled gas from the patient also reaches the expiratory branch through the connection pipeline of the breathing circuit.

In one embodiment, the anesthesia machine is provided with one or more of a gas source module, a common gas outlet and a fresh gas outlet, and when the oxygen battery is being calibrated, the gas source module, the common gas outlet or the fresh gas outlet supplies the calibration gas to the inspiratory branch.

In one embodiment, further including an expiratory device, which is arranged between the expiratory branch and the exhaust gas discharge device for adjusting the flow rate or pressure of the exhaled gas.

In one embodiment, the anesthesia machine further includes a controller, which controls the oxygen battery calibration system to perform the oxygen battery calibration during self-test of the anesthesia machine.

In one embodiment, the self-test of the anesthesia machine further includes gas tightness detection or backup flow control system test.

The present disclosure also relates to a calibration method of an oxygen battery calibration system, wherein the calibration method is applied to the oxygen battery calibration system, the oxygen battery calibration system including the breathing circuit and the oxygen battery, wherein the breathing circuit includes the inspiratory branch, the expiratory branch and the connection pipeline, and the oxygen battery is connected to the inspiratory branch, with the joint being located at the rear end of the inspiratory one-way valve. The calibration method can include controlling the calibration gas to enter the inspiratory branch and flow out through the oxygen battery, the connection pipeline and the expiratory branch. The calibration method can also include performing an oxygen concentration calibration according to the calibration gas flowing through the oxygen battery.

With the use of the above technical solutions, the present disclosure in certain embodiments has the following beneficial effects. The anesthesia machine, the oxygen battery calibration system and the calibration method thereof of the present disclosure may realize automatic calibration of an oxygen battery without manual intervention, ensure the reliability the operation of the oxygen battery, and enable the anesthesia machine to operate normally.

### BRIEF DESCRIPTION OF THE DRAWINGS

FIG. 1 is a schematic diagram of an oxygen battery calibration system of an embodiment of the present disclosure;

FIG. 2 is a schematic diagram of an implementation of an oxygen battery calibration system of another embodiment of the present disclosure;

FIG. 3 is a schematic diagram of another implementation of the oxygen battery calibration system shown in FIG. 2; and

FIG. 4 is a schematic diagram of a gas path of an anesthesia machine of another embodiment of the present disclosure.

In the figures:
100—Oxygen battery calibration system;
110—Breathing circuit;
111—Inspiratory branch;
112—Expiratory branch;
113—Absorption tank branch;
114—Inspiratory one-way valve;
115—Expiratory one-way valve;
116—Connection pipeline;
117—CO₂ absorption tank;
120—Oxygen battery;
130—Bypass branch;
140—Switch component;
200—Anesthetic supply device;
300—Expiratory device;
310—Expiratory pipeline;
320—Expiratory valve;
330—Adjustment valve;
340—Adjustment pipeline.

### DETAILED DESCRIPTION

To make the objectives, technical solutions, and advantages of the present disclosure more apparent, the anesthesia machine, the oxygen battery calibration system and the calibration method thereof of the present disclosure are described below in further detail by way of embodiments and with reference to the accompanying drawings. It should be understood that the particular embodiments described herein are merely intended to explain the present disclosure and is not intended to define the present disclosure.

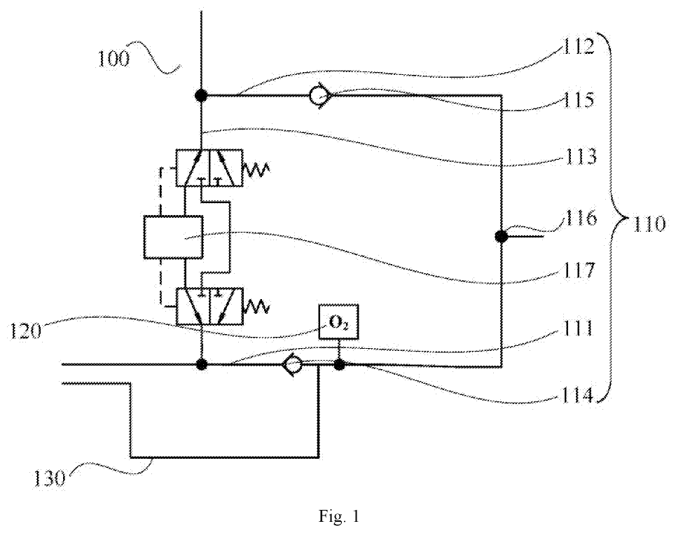

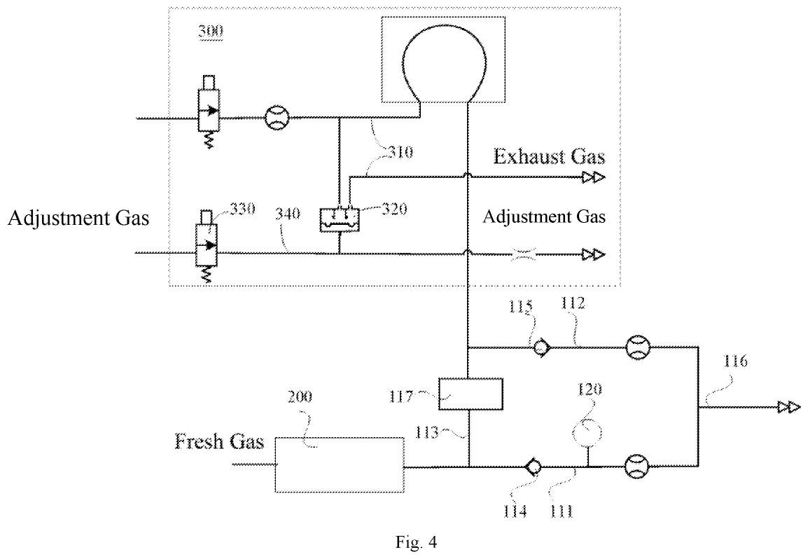

Referring to FIGS. 1 to 3, the present disclosure provides an oxygen battery calibration system 100. The oxygen battery calibration system 100 includes a breathing circuit 110, an oxygen battery 120, and a calibration management controller. The oxygen battery calibration system 100 may be used in an anesthesia machine shown in FIG. 4. The oxygen battery calibration system 100 is applied in the anesthesia machine and used to calibrate the oxygen battery 120 of the anesthesia machine. In this way, the oxygen battery 120 may reliably detect the oxygen concentration in the gas in the breathing circuit 110 when the anesthesia machine is in use, such that the oxygen concentration in the gas supplied to a patient may meet actual demands and the use safety of the patient is ensured. If the oxygen battery 120 needs to be calibrated, control a calibration gas flowing through the oxygen battery 120 to perform the oxygen concentration calibration. The oxygen battery 120 here refers to a medical oxygen battery. Of course, in other implementations of the present disclosure, the oxygen battery calibration system 100 of the present disclosure may also be used in the oxygen concentration calibration of the oxygen battery 120 in other apparatuses, such as a ventilator.

In the present disclosure, the breathing circuit 110 includes an inspiratory branch 111, an expiratory branch 112, an absorption tank branch 113, an inspiratory one-way valve 114, a connection line 116, and an expiratory one-way valve 115. The inspiratory branch 111 communicates with the expiratory branch 112 through the connection pipeline 116. The inspiratory one-way valve 114 is arranged in the inspiratory branch 111, and the expiratory one-way valve 115 is arranged in the expiratory branch 112. One end of the absorption tank branch 113 communicates with the inspiratory branch 111, and the one end of the absorption tank branch 113 is located at a front end of the inspiratory one-way valve 114. The other end of the absorption tank branch 113 communicates with the expiratory branch 112 and is located at a rear end of the expiratory one-way valve 115. The oxygen battery 120 is connected to the inspiratory branch 111, with a joint being located at a rear end of the inspiratory one-way valve 114.

During normal use of the anesthesia machine, a first end of the breathing circuit 110 communicates with an anesthetic supply device 200, a second end of the breathing circuit 110 communicates with an exhaust gas discharge device, and the breathing circuit 110 also communicates with a breathing end of the patient. The first end here refers to a gas inlet end of the breathing circuit 110, and the second end refers to a gas outlet end of the breathing circuit 110. The anesthetic supply device 200 supplies an inspiratory gas containing an anesthetic to the breathing circuit 110 and delivers the gas to the patient. The exhaled gas containing the anesthetic exhaled from the patient is purified in the circuit by a CO₂ absorption tank 117 and is then reused, and the remaining gas is processed by the exhaust gas discharge device. Moreover, the exhaust gas discharge device may be either an exhaust gas discharge device or an exhaust gas recovery device, or may further be another device capable of processing exhaust gas.

Specifically, a gas inlet end of the inspiratory branch 111 is capable of communicating with the anesthetic supply device 200 of the anesthesia machine. The connection pipeline 116 is used to connect a gas outlet end of the inspiratory branch 111, a gas inlet end of the expiratory branch 112 and the breathing end of the patient. A gas outlet end of the expiratory branch 112 communicates with the exhaust gas discharge device. Here, the gas inlet end of the inspiratory branch 111 coincides with the gas inlet end of the breathing circuit 110 and they are the same end, and the gas outlet end of the expiratory branch 112 coincides with the gas outlet end of the breathing circuit 110 and they are the same end. The inspiratory gas containing the anesthetic delivered by the anesthetic supply device enters the inspiratory branch 111, passes through the inspiratory branch 111 and then enters the breathing end of the patient through the connection pipeline 116 to supply the patient with the anesthetic. The exhaled gas from the patient enters the connection pipeline 116 via the breathing end, and enters the expiratory branch 112 through the connection pipeline 116. In an inhalation phase, the exhaled gas passes through the CO₂ absorption tank 117, and is reused after CO₂ is absorbed, and the remaining gas is discharged from the exhaust gas discharge device via an expiratory valve. Moreover, the connection pipeline 116 includes a Y-type pipe or a communicating pipe for connecting the inspiratory branch 111 and the expiratory branch 112, and ends of the Y-type pipe are respectively connected to the inspiratory branch 111, the expiratory branch 112 and the patient. The Y-type pipe in capable of facilitating the inspiratory gas to enter the breathing end of the patient through the inspiratory branch 111, and also allows the exhaled gas from the patient to enter the expiratory branch 112. Also, the inspiratory one-way valve 114 is arranged in the inspiratory branch 111, which may prevent the inspiratory gas flowing through the inspiratory one-way valve 114 from returning, so that the inspiratory gas flows in a single direction. The expiratory one-way valve 115 is arranged in the expiratory branch 112, which may prevent the exhaled gas flowing through the expiratory one-way valve 115 from returning, so that the exhaled gas flows in a single direction. The inspiratory one-way valve 114 is located downstream of the joint between the inspiratory branch 111 and the absorption tank branch 113, that is, the inspiratory gas first passes through the joint between the inspiratory branch 111 and the absorption tank branch 113 in the inspiratory branch 111 and then flows through the inspiratory one-way valve 114. The expiratory one-way valve 115 is located upstream of the joint between the expiratory branch 112 and the absorption tank branch 113, that is, the exhaled gas first passes through the expiratory one-way valve 115 in the expiratory branch 112 and then flows through the joint between the expiratory branch 112 and the absorption tank branch 113.

The oxygen battery 120 is used to detect whether the oxygen concentration in the inspiratory gas delivered by the anesthesia machine reaches a standard, specifically: the oxygen battery 120 is capable of detecting the oxygen concentration in the inspiratory gas delivered to the patient through the inspiratory branch 111 to ensure that the oxygen concentration in the inspiratory gas containing the anesthetic supplied to the patient may meet the demands and ensure the safety during use. If the oxygen concentration in the inspiratory gas containing the anesthetic is lower or higher than a pre-set oxygen concentration for the anesthesia machine, it may cause potential hazards to the safety of the patient. After the oxygen battery 120 has been used for a period of time, there may be a certain deviation between the oxygen concentration detected by the oxygen battery 120 and the actual oxygen concentration. Therefore, the oxygen battery 120 needs to be calibrated regularly or as required to ensure that the oxygen battery 120 may accurately detect the oxygen concentration in the inspiratory gas, to ensure the reliability of detection of the oxygen concentration, and in turn to ensure the reliable operation of the anesthesia machine.

Moreover, the oxygen concentration calibration of the oxygen battery 120 is achieved by using a calibration gas, and the calibration management controller is used to control the flow of the calibration gas. If the oxygen battery 120 needs to be calibrated, the calibration management controller controls the calibration gas to enter the inspiratory branch 111 and pass through the oxygen battery 120, the connection pipeline 116, and the expiratory branch 112. The calibration management controller performs the oxygen concentration calibration according to the calibration gas flowing through the oxygen battery 120. Specifically, the calibration management controller collects the current output by the oxygen battery 120 that corresponds to the oxygen content in the calibration gas to obtain a linear function relationship between the oxygen concentration and the output current. The calibration management controller may further store the obtained linear function relation for the oxygen concentration in a memory of the anesthesia machine. During a subsequent oxygen concentration measurement, a controller of the anesthesia machine may obtain the oxygen concentration of the inspected gas by means of the reverse calculation from the value of the current output from the oxygen battery 120 according to the linear function relationship for the oxygen concentration stored in the memory. In a specific embodiment, the controller of the anesthesia machine and the calibration management controller may be either two different components or the same component, for example a board integrated with software algorithms and a hardware controller.

Specifically, if the oxygen battery 120 is a chemical oxygen battery, the calibration management controller may calibrate the oxygen battery 120 using a calibration gas having at least two different oxygen concentrations. In this way, the accuracy of the concentration calibration of the oxygen battery 120 may be ensured and the safety coefficient during use is improved. In an implementation of the present disclosure, a calibration gas having an oxygen concentration of 21% and a calibration gas having an oxygen concentration of 100% are respectively introduced into the breathing circuit 110. When the oxygen battery 120 is being calibrated, firstly, the calibration gas having the oxygen concentration of 21% is introduced into the inspiratory branch 111 at a flow rate of 5 L/min for 1 minute to stabilize the value of the current output by the oxygen battery 120, and the calibration management controller collects and stores the current value of the current output by the oxygen battery 120; secondly, the calibration gas having the oxygen concentration of 100% is introduced into the inspiratory branch 111 at a flow rate of 5 L/min for 1 minute to stabilize the value of the current output by the oxygen battery 120, the calibration management controller samples the current value of the current output by the oxygen battery 120 and stores same in the memory. The calibration management controller obtains a linear function relation for the oxygen concentration according to the corresponding relationship between the values of the current and the values of the oxygen concentrations at two calibration points, and stores same in the memory, completing the calibration operation of the oxygen battery 120. Of course, the oxygen concentrations of the calibration gas introduced into the inspiratory branch 111 may also be selected to be any other two controllable oxygen concentrations, such as 30% and 90%, etc. Of course, in addition to the calibration gases having the above-mentioned oxygen concentrations of 21% and 100% introduced into the breathing circuit 110, the calibration gas having one of more of the oxygen concentrations of 30%, 40% and 90% may also be introduced. Furthermore, if the requirements for calibration effects are not too high, the calibration may also be performed by using only the calibration gas having one oxygen concentration.

Of course, in another implementation of the present disclosure, the oxygen battery 120 is a paramagnetic oxygen battery, and the calibration management controller calibrates the oxygen battery 120 with the calibration gas having at least one oxygen concentration. The paramagnetic oxygen battery may be calibrated with the calibration gas having only one oxygen concentration, or may also be calibrated with the calibration gas having two or more oxygen concentrations. The calibration gas having the oxygen concentration of 21% or the calibration gas having the oxygen concentration of 100% is introduced into the inspiratory branch 111. When the oxygen battery 120 is being calibrated, the calibration gas having the oxygen concentration of 21% is introduced into the breathing circuit 110 at the flow rate of 5 L/min for 1 minute to stabilize the current output by the oxygen battery 120, the calibration management controller collects and stores the current value of the current output by the oxygen battery 120, and the calibration management controller samples and stores the current value of the current output by the oxygen battery 120 in the memory. Alternatively, the calibration gas having the oxygen concentration of 100% is introduced into the breathing circuit 110 at the flow rate of 5 L/min for 1 minute to stabilize the current output by the oxygen battery 120, the calibration management controller collects and stores the current value of the current output by the oxygen battery 120, and the calibration management controller samples and stores the current value of the current output by the oxygen battery 120 in the memory. The controller draws a linear function relation according to the corresponding relationship between the value of the current and the oxygen concentration value at the calibration point and stores same in the memory, completing the calibration operation of the oxygen battery 120. Of course, the calibration gas having the oxygen concentration of 21% and the calibration gas having the oxygen concentration of 100% may also be respectively introduced into the inspiratory branch 111. Of course, the oxygen concentration of the calibration gas introduced into the inspiratory branch 110 may also be selected to be any one or two other controllable oxygen concentrations, such as 30% and 90%. Of course, in addition to the calibration gases having the above-mentioned oxygen concentrations of 21% and 100% introduced into the breathing circuit 110, the calibration gas having one of more of the oxygen concentrations of 30%, 40% and 90% may also be introduced.

In the above calibration operation, the flow rate and the period of time of the calibration gas introduced into the inspiratory branch 111 may also be set according to the specific structure and volume of the breathing circuit, for example, flow rate of 5 L/min for 3 min, flow rate of 8 L/min for 2 min, flow rate of 10 L/min for 1 min, etc. The relationship between the flow rate and the period of time is aimed to completely replace and stabilize the types of gases in a measurement area of the oxygen battery.

During the calibration of the oxygen battery 120, the anesthetic supply device 200 is required to be turned off, such that the calibration gas provided by the anesthesia machine enters the inspiratory branch 111, and the calibration gas is input into the inspiratory branch 111. The calibration management controller controls the calibration gas to enter the inspiratory branch 111 and flow out through the oxygen battery 120, the connection pipeline 116 and the expiratory branch 112. The calibration management controller performs the oxygen concentration calibration according to the calibration gas flowing through the oxygen battery 120. When the calibration gas flows through the oxygen battery 120, the oxygen battery 120 may output a corresponding value of the current according to the value of the actual oxygen concentration in the calibration gas, and the calibration management controller stores the obtained function relation between the values of the oxygen concentration and the current in the memory to realize the calibration operation of the oxygen battery 120.

The oxygen battery calibration system 100 of the present disclosure enables the calibration gas to flow through the rear end of the inspiratory one-way valve 114 in the inspiratory branch 111, pass through the connection pipeline 116 and then flow out through the expiratory branch 112. During the calibration of the oxygen battery 120, the calibration management controller controls the calibration gas to directly enter the expiratory branch 112 through the inspiratory branch 111, without disconnecting the connection pipeline 116. Since the oxygen battery 120 is connected to the inspiratory branch 111 at the rear end of the inspiratory one-way valve 114, when the calibration gas flows in the inspiratory branch 111, replacement with the calibration gas in the inspiratory branch 111 may occur in the measurement area of the oxygen battery 120, such that automatic calibration of the oxygen battery 120 is realized to ensure the reliability of the operation of the oxygen battery 120 and to enable the anesthesia machine to operate normally. Several ways in which the calibration gas enters the connection pipeline 116 via the rear end of the inspiratory one-way valve 114 are described in detail as follows.

Referring to FIG. 1, in an embodiment of the present disclosure, the oxygen battery calibration system 100 further includes a bypass branch 130, and the bypass branch 130 and the inspiratory branch 111 are connected between the inspiratory one-way valve 114 and the oxygen battery 120. The calibration management controller controls the calibration gas to enter the inspiratory branch 111 through the bypass branch 130 during the calibration. That is to say, one bypass branch 130 is separately provided, and the bypass branch 130 is directly introduced into the inspiratory branch 111 at the rear end of the inspiratory one-way valve 114 and is located before the oxygen battery 120, namely the calibration gas enters the inspiratory branch 111 through the bypass branch 130 and then flows through the oxygen battery 120. Moreover, thanks to the effect of the inspiratory one-way valve 114, the calibration gas may only flow along the inspiratory branch 111 through the oxygen battery 120 and the connection pipeline 116 into the expiratory branch 112, which may prevent the calibration gas from entering the absorption tank branch 113. When the oxygen battery 120 is being calibrated, the calibration management controller controls the calibration gas to enter the inspiratory branch 111 through the bypass branch 130, and flow through the oxygen battery 120 and the connection pipeline 116 into the expiratory branch 112, such that the automatic calibration of the oxygen battery 120 is realized to ensure the reliability of the operation of the oxygen battery 120 and to enable the anesthesia machine to operate normally.

Further, one end of the bypass branch 130 is connected to a common gas outlet or a fresh gas outlet of the anesthesia machine and the other end thereof is connected to the rear end of the inspiratory one-way valve in the inspiratory branch 111. It may be understood that both the common gas outlet and the fresh gas outlet may deliver the calibration gas. The calibration management controller controls the calibration gas to flow from the common gas outlet or the fresh gas outlet into the bypass branch 130, then pass through the bypass branch 130 into the inspiratory branch 111, and flow through the oxygen battery 120 and the connection pipeline 116 into the expiratory branch 112, such that the automatic calibration of the oxygen battery 120 is realized to ensure the reliability of the operation of the oxygen battery 120 and to enable the anesthesia machine to operate normally.

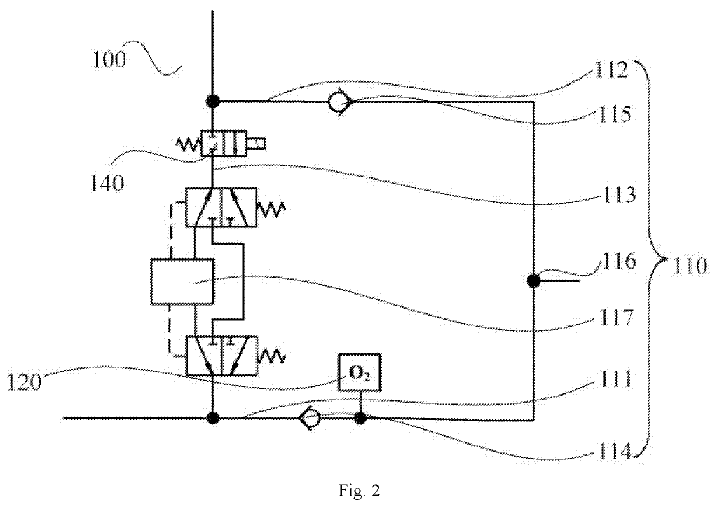

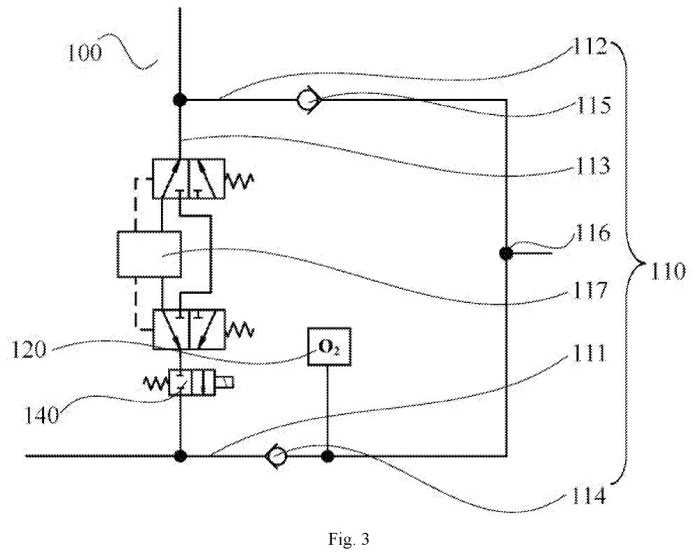

Referring to FIGS. 2 and 3, in another embodiment of the present disclosure, the oxygen battery calibration system 100 further includes a switch component 140. The switch component 140 is arranged in the absorption tank branch 113 and is used to control the opening and closing of the absorption tank branch 113. When the oxygen concentration calibration is performed, the switch component 140 closes the absorption tank branch 113 and the calibration gas may enter the inspiratory branch 111. Since the switch component 140 closes the absorption tank branch 113, when the calibration gas flows in the inspiratory branch 111, the calibration gas may not flow along the absorption tank branch 113 but may only continue to flow along the inspiratory branch 111, and may flow from the front end to the rear end of the inspiratory one-way valve 114 and flow through the oxygen battery 120 and the connection pipeline 116 into the expiratory branch 112, such that the automatic calibration of the oxygen battery 120 is realized to ensure the reliability of the operation of the oxygen battery 120 and to enable the anesthesia machine to operate normally.

For example, the switch component 140 may be a switch valve, an air-resistor, or another element capable of closing the absorption tank branch or preventing a large amount of gas from flowing through the absorption tank branch. In this way, all or most of the calibration gas may flow into the inspiratory branch 111 and pass through the connection line 116 and then flow out through the expiratory branch 112. Moreover, the switch component 140 may be arranged between the CO₂ absorption tank 117 and the inspiratory branch 111, as shown in FIG. 3, or may be also be arranged between the CO₂ absorption tank 117 and the expiratory branch 112, as shown in FIG. 2, as long as it may be ensured that the switch component 140 closes the absorption tank branch 113 or provides sufficient gas flow resistance in the absorption tank branch 113.

At this point, an input end of the inspiratory branch 111 may communicate with a gas source module of the anesthesia machine, or the common gas outlet, or the fresh gas outlet, and the calibration management controller controls the calibration gas to enter the inspiratory branch 111 during the oxygen concentration calibration. It may be understood that each of the gas source module, the common gas outlet and the fresh gas outlet may deliver the calibration gas. The calibration management controller controls the calibration gas to flow from the gas source module, the common gas outlet or the fresh gas outlet into the inspiratory branch 111, and flow through inspiratory one-way valve 114, the oxygen battery 120 and the connection pipeline 116 into the expiratory branch 112, such that the automatic calibration of the oxygen battery 120 is realized to ensure the reliability of the operation of the oxygen battery 120 and to enable the anesthesia machine to operate normally.

Of course, in other embodiments of the present disclosure, the flow rate of the calibration gas may be adjusted by a flow meter or a control valve, etc., and then the calibration gas enters the bypass branch 130, and then enters the inspiratory branch 111 via the rear end of the inspiratory one-way valve 114. Alternatively, after the absorption tank branch 113 is closed by the switch component 140, the flow rate of the calibration gas is adjusted by the flow meter or the control valve, etc., and then the calibration gas enters the inspiratory branch 111. It should be noted that the communication between the various components of the calibration system for the oxygen battery 120 is achieved through pipelines or by means of direct assembly and sealing. While the components described in some sections are directly connected to the inspiratory branch 111, the expiratory branch 112, etc., the components are actually connected to the inspiratory branch 111, the expiratory branch 112, etc. via the pipelines or by means of direct assembly and sealing, which are not described in detail here. Optionally, an inspiratory flow sensor is arranged in the inspiratory branch 111 for detecting the flow rate of the inspiratory gas in the inspiratory branch 111 to prevent a too large or too low flow rate of the inspiratory gas to ensure safe use. An expiratory flow sensor is arranged in the expiratory branch 112 for detecting the flow of the exhaled gas in the expiratory branch 112 to prevent a too large or too low flow rate of the exhaled gas to ensure safe use.

Referring to FIG. 4, the present disclosure further provides an anesthesia machine including an anesthetic supply device 200, an exhaust gas discharge device (not shown), and the oxygen battery calibration system 100 as in the above embodiment. The gas inlet end of the inspiratory branch 111 of the breathing circuit 110 of the oxygen battery calibration system 100 communicates with the anesthetic supply device 200, and the gas outlet end of the expiratory branch 112 of the breathing circuit 110 communicates with the exhaust gas discharge device via an expiratory valve. When the anesthesia machine is in use, the anesthetic supply device 200 supplies the inspiratory gas containing the anesthetic to the breathing circuit 110, the inspiratory gas enters the inspiratory branch 111 and is then supplied to the breathing end of the patient through the connection pipeline 116, and at the same time, the exhaled gas from the patient may also be delivered out of the breathing end of the patient. The exhaled gas is reused after CO₂ is absorbed by the CO₂ absorption tank 117 in the breathing circuit 110. The remaining exhaled gas is purified by the exhaust gas discharge device after passing through the expiratory valve 320. In this way, pollutions caused by direct discharge into the atmosphere may be prevented while effects on medical personnel may be prevented.

The anesthesia machine also has one or more of the gas source module, the common gas outlet, and the fresh gas outlet. When the oxygen battery 120 is being calibrated, the calibration gas may enter the inspiratory branch 111 through the gas source module, the common gas outlet or the fresh gas outlet, and flow through the oxygen battery 120 and the connection pipeline 116 into the expiratory branch 112, such that automatic calibration of the oxygen battery 120 is realized to ensure the reliability of the operation of the oxygen battery 120 and to enable the anesthesia machine to operate normally.

Further, the anesthesia machine further includes an expiratory device 300. The expiratory device 300 is arranged between the expiratory branch 112 and the exhaust gas discharge device for adjusting the pressure or flow rate of the exhaled gas. The expiratory device 300 includes a pressure adjustment structure and the expiratory valve 320. The gas inlet end and the gas outlet end of the expiratory valve 320 are respectively connected to an expiratory pipeline 310. One end of the expiratory valve 320 is connected to one end of the expiratory branch 112 through the expiratory pipeline 310, and the other end of the expiratory valve communicates with the exhaust gas discharge device through the expiratory pipeline 310. The expiratory valve 320 is used to discharge the exhaled gas from the patient, and the exhaled gas from the patient may enter the expiratory valve 320 through the expiratory branch 112 and through the expiratory pipeline 310, and is then discharged after being adjusted in pressure by the expiratory valve 320. A pressure control structure is used to adjust the flow rate or pressure of the exhaled gas delivered out of the expiratory valve 320 to adjust the valve closing pressure of the expiratory valve 320 to achieve the purpose of closing the expiratory valve 320 at a set pressure. In this way, if the inspiratory pressure in the inspiratory branch 111 of the breathing circuit 110 exceeds the valve closing pressure of the expiratory valve, the remaining inspiratory gas in the inspiratory branch 111 will directly flow into the expiratory valve 320 for release without reaching the patient, thereby ensuring that the pressure of the inspiratory gas at a patient end will not exceed the set pressure, so as to ensure the safety of the patient during use and improve the safety coefficient. The pressure control structure may be an adjustable pressure limitation valve (APL valve for short) or another structure capable of adjusting the pressure of the exhaled gas. In this embodiment, the pressure control structure includes an adjustment pipeline 340 for inputting and outputting an adjustment gas and an adjustment valve 330 arranged in the adjustment pipeline 340. An output end of the adjustment valve 330 communicates with the expiratory valve 320. The adjustment valve 330 may adjust the pressure or flow rate of the pressure adjustment gas in the adjustment pipeline 340 to achieve the pressure adjustment of the exhaled gas in the expiratory valve 320.

With the advancement of science and technology, many self-test items of the anesthesia machine, such as gas tightness, flow measurement accuracy, and flow sensor calibration, have been fully automated, which not only frees up the medical personnels to enable them to focus more on patients and surgical procedures, but also improves the reliability of equipment and avoids deviations caused by manual intervention.

Further, the anesthesia machine further includes a controller, which controls the oxygen battery calibration system to perform the oxygen battery calibration during the self-test of the anesthesia machine. The self-test of the anesthesia machine includes one or more of gas tightness detection, backup flow control system detection or flow sensor calibration, etc. The controller may control the anesthesia machine to carry out the self-test when being started up and in standby, or the anesthesia machine may also carry out the self-test regularly or according to a control signal input by a user.

The calibration of the oxygen battery is completed together with the self-test of the anesthesia machine, which does not require operator intervention or concern at all, thereby reducing the operator's burden and preventing potential safety hazards to the patient due to inaccurate measurement of the oxygen concentration during use of the anesthesia machine caused by the operator forgetting to perform the calibration. Further, completing the calibration of the oxygen battery and the self-test for the air tightness of the anesthesia machine together may also eliminate the operation of closing the connection pipeline by the operator.

The present disclosure further provides a calibration method of an oxygen battery calibration system. The calibration method is applied to the above-mentioned oxygen battery calibration system 100. The calibration method includes the following steps: controlling the calibration gas to enter the inspiratory branch 111 and flow out through the oxygen battery 120, the connection pipeline 116 and the expiratory branch 112; and performing the oxygen concentration calibration according to the calibration gas flowing through the oxygen battery 120.

When the oxygen battery 120 is being calibrated, the calibration management controller controls the calibration gas to enter the inspiratory branch 111 and flow out through the oxygen battery 120, the connection pipeline 116, and the expiratory branch 112 without closing the connection pipeline 116. The calibration management controller performs the oxygen concentration calibration according to the calibration gas flowing through the oxygen battery 120 to realize the automatic calibration of the oxygen battery 120. Moreover, when the calibration gas flows through the oxygen battery 120, the oxygen battery 120 may output a corresponding value of the current according to the value of the actual oxygen concentration in the calibration gas, and the controller stores the function relation between the values of the oxygen concentration and the current to realize the calibration operation of the oxygen battery 120.

The various technical features of the embodiments described above can be arbitrarily combined. In order to simplify the description, all possible combinations of the various technical features in the above embodiments have not been described. However, any combination of these technical features should be considered to fall within the scope of the disclosure of this description as long as there is no contradiction.

The above-mentioned embodiments merely represent several implementations of the present disclosure, giving specifics and details thereof, but should not be understood as limiting the scope of the present patent of disclosure thereby. It should be noted that a person of ordinary skill in the art could also make several variations and improvements without departing from the concept of the present disclosure. These variations and improvements all fall within the scope of protection of the present disclosure. Therefore, the scope of protection of the present patent of disclosure shall be in accordance with the appended claims.

What is claimed is:

1. An oxygen battery calibration system, comprising:
a breathing circuit comprising an inspiratory branch, an expiratory branch, an absorption tank branch, an inspiratory one-way valve, a connection pipeline and an expiratory one-way valve, wherein the inspiratory branch and the expiratory branch communicate via the connection pipeline, the inspiratory one-way valve is arranged in the inspiratory branch, the expiratory one-way valve is arranged in the expiratory branch, one end of the absorption tank branch communicates with the inspiratory branch and is located at a front end of the inspiratory one-way valve, and the other end of the absorption tank branch communicates with the expiratory branch and is located at a rear end of the expiratory one-way valve;
an oxygen battery connected to the inspiratory branch, with a joint being located at a rear end of the inspiratory one-way valve; and
a calibration management controller, the calibration management controller controlling a calibration gas to enter the inspiratory branch and flow out through the oxygen battery, the connection pipeline and the expiratory branch, and the calibration management controller performing an oxygen concentration calibration according to the calibration gas flowing through the oxygen battery.

2. The oxygen battery calibration system of claim 1, further comprising a bypass branch, wherein the bypass branch and the inspiratory branch are connected between the inspiratory one-way valve and the oxygen battery; and
the calibration management controller controls the calibration gas to enter the inspiratory branch through the bypass branch during the oxygen concentration calibration.

3. The oxygen battery calibration system of claim 1, further comprising a switch component, wherein the switch component is arranged in the absorption tank branch for controlling the opening and closing of the absorption tank branch; and
when the oxygen concentration calibration is performed, the switch component closes the absorption tank branch and the calibration gas is capable of entering the inspiratory branch.

4. The oxygen battery calibration system of claim 3, wherein the switch component is a switch valve or an air-resistor.

5. The oxygen battery calibration system of claim 2, wherein one end of the bypass branch is connected to a common gas outlet or a fresh gas outlet of an anesthesia machine, and the other end thereof is connected to the rear end of the inspiratory one-way valve in the inspiratory branch.

6. The oxygen battery calibration system of claim 3, wherein an input end of the inspiratory branch communicates with a gas source module of an anesthesia machine, a common gas outlet, or a fresh gas outlet, and the calibration management controller controls the calibration gas to enter the inspiratory branch during the oxygen concentration calibration.

7. The oxygen battery calibration system of claim 1, wherein the oxygen battery is a chemical oxygen battery, and the calibration management controller calibrates the oxygen battery with the calibration gas having at least two different oxygen concentrations; or the oxygen battery is a paramagnetic oxygen battery, and the calibration management controller calibrates the oxygen battery with the calibration gas having at least one oxygen concentration.

8. An anesthesia machine, comprising an anesthetic supply device, an exhaust gas discharge device, and an oxygen battery calibration system, wherein the oxygen battery calibration system comprises:
a breathing circuit comprising an inspiratory branch, an expiratory branch, an absorption tank branch, an inspiratory one-way valve, a connection pipeline and an expiratory one-way valve, wherein the inspiratory branch and the expiratory branch communicate via the connection pipeline, the inspiratory one-way valve is arranged in the inspiratory branch, the expiratory one-way valve is arranged in the expiratory branch, one end of the absorption tank branch communicates with the inspiratory branch and is located at a front end of the inspiratory one-way valve, and the other end of the absorption tank branch communicates with the expiratory branch and is located at a rear end of the expiratory one-way valve;
an oxygen battery connected to the inspiratory branch, with a joint being located at a rear end of the inspiratory one-way valve; and
a calibration management controller, the calibration management controller controlling a calibration gas to enter the inspiratory branch and flow out through the oxygen battery, the connection pipeline and the expiratory branch, and the calibration management controller performing an oxygen concentration calibration according to the calibration gas flowing through the oxygen battery;
one end of the inspiratory branch of the breathing circuit of the oxygen battery calibration system communicates with the anesthetic supply device, and one end of the expiratory branch of the breathing circuit communicates with the exhaust gas discharge device; and
during use of the anesthesia machine, the anesthetic supply device supplies an inspiratory gas containing an anesthetic to the breathing circuit, the inspiratory gas enters the inspiratory branch and is then supplied to a patient through the connection pipeline, and at the same time, an exhaled gas from the patient also reaches the expiratory branch through the connection pipeline of the breathing circuit.

9. The anesthesia machine of claim 8, wherein the anesthesia machine is provided with one or more of a gas source module, a common gas outlet and a fresh gas outlet, and when the oxygen battery is being calibrated, the gas source module, the common gas outlet or the fresh gas outlet supplies the calibration gas to the inspiratory branch.

10. The anesthesia machine of claim 9, wherein the oxygen battery calibration system further comprises a bypass branch, the bypass branch and the inspiratory branch are connected between the inspiratory one-way valve and the oxygen battery;
one end of the bypass branch is connected to the common gas outlet or the fresh gas outlet of the anesthesia machine, and the other end thereof is connected to the rear end of the inspiratory one-way valve in the inspiratory branch; and
the calibration management controller controls the calibration gas to enter the inspiratory branch through the bypass branch during the oxygen concentration calibration.

11. The anesthesia machine of claim 9, wherein the oxygen battery calibration system further comprises a switch component, the switch component is arranged in the absorption tank branch for controlling the opening and closing of the absorption tank branch; and
when the oxygen concentration calibration is performed, the switch component closes the absorption tank branch and permits the calibration gas to enter the inspiratory branch.

12. The anesthesia machine of claim 9, wherein the oxygen battery is a chemical oxygen battery, and the calibration management controller calibrates the oxygen battery with the calibration gas having at least two different oxygen concentrations; or the oxygen battery is a paramagnetic oxygen battery, and the calibration management controller calibrates the oxygen battery with the calibration gas having at least one oxygen concentration.

13. The anesthesia machine of claim 8, further comprising an expiratory device, which is arranged between the expiratory branch and the exhaust gas discharge device for adjusting the flow rate or pressure of the exhaled gas.

14. The anesthesia machine of claim 8, further comprising a controller, which controls the oxygen battery calibration system to perform the oxygen battery calibration during self-test of the anesthesia machine.

15. The anesthesia machine of claim 14, wherein the self-test of the anesthesia machine further comprises one or more of gas tightness detection, flow control system test or flow sensor calibration.

16. A calibration method of an oxygen battery calibration system, wherein the calibration method is applied to an oxygen battery calibration system, the oxygen battery calibration system comprises:
a breathing circuit comprising an inspiratory branch, an expiratory branch, an absorption tank branch, an inspiratory one-way valve, a connection pipeline and an expiratory one-way valve, wherein the inspiratory branch and the expiratory branch communicate via the connection pipeline, the inspiratory one-way valve is arranged in the inspiratory branch, the expiratory one-way valve is arranged in the expiratory branch, one end of the absorption tank branch communicates with the inspiratory branch and is located at a front end of the inspiratory one-way valve, and the other end of the absorption tank branch communicates with the expiratory branch and is located at a rear end of the expiratory one-way valve;
an oxygen battery connected to the inspiratory branch, with a joint being located at a rear end of the inspiratory one-way valve; and
the calibration method comprises:
controlling the calibration gas to enter the inspiratory branch and flow out through the oxygen battery, the connection pipeline and the expiratory branch; and
performing an oxygen concentration calibration according to the calibration gas flowing through the oxygen battery.

17. The calibration method of claim 16, wherein the calibration method is applied to an oxygen battery calibration system which is arranged in an anesthesia machine, and the calibration method is performed during self-test of the anesthesia machine.

18. The calibration method of claim 16, wherein the oxygen battery calibration system further comprises a bypass branch, the bypass branch and the inspiratory branch are connected between the inspiratory one-way valve and the oxygen battery; and the controlling the calibration gas to enter the inspiratory branch comprises: controlling the calibration gas to enter the inspiratory through the bypass branch.

19. The calibration method of claim 16, wherein the oxygen battery calibration system further comprises a switch component, the switch component is arranged in the absorption tank branch for controlling the opening and closing of the absorption tank branch; and the calibration method further comprises:
the switch component closes the absorption tank branch and the calibration gas is capable of entering the inspiratory branch.

20. The calibration method of claim 16, wherein the calibration method further comprises: calibrates the oxygen battery with the calibration gas having at least two different oxygen concentrations
